(19) United States
(12) Patent Application Publication
(10) Pub. No.: US 2022/0208345 A1
(43) Pub. Date: Jun. 30, 2022

(54) METHOD AND SYSTEM OF ARRANGING AND DELIVERING A NUTRITIONALLY PERSONALIZED MEAL PORTION

(71) Applicant: Madjid Mohimani, Irvine, CA (US)

(72) Inventor: Madjid Mohimani, Irvine, CA (US)

(21) Appl. No.: 17/351,829

(22) Filed: Jun. 18, 2021

Related U.S. Application Data
(60) Provisional application No. 63/133,022, filed on Dec. 31, 2020.

(51) Int. Cl. G16H 20/60; G16H 50/70; G06Q 10/08; G16H 40/20; G06Q 30/06; G06Q 50/12; A23L 33/10

(52) U.S. Cl. CPC G16H 20/60; G16H 50/70; G06Q 10/083; A23V 2002/00; G06Q 30/0621; G06Q 50/12; A23L 33/10; G16H 40/20

(57) ABSTRACT

The present invention discloses a method and/or system to arrange production and delivery of personalized or healthy meal portions to consumers/users. A specific recipe input from a user is received to select at least one healthy ingredient from a plurality of healthy ingredients and personalize healthy meal portions based on the user's personal taste and choices.

### FIELD OF THE INVENTION

The present invention relates to a method and/or system of arranging delivery of meal portions to consumers. The present invention particularly relates to delivery of personalized meal portions to consumers/users.

### BACKGROUND OF THE INVENTION

There are various entities in the food and beverage industry that attempt to target consumers with nutritionally “healthy” foods, drinks, supplements, or other nutritional products. “Healthy” foods or food products are those foods that have ingredients designed to deliver necessary nutrients in a balanced manner, while also reducing toxic or bad substances such as pesticides or saturated fats. Consumers are also experiencing a wide variety of online and offline options to purchase these types of products. However, many such products are either targeted to a specific purpose or demographic, or to a large population in general. For example, various energy drinks or supplements are available in the market to enhance performance of a generic consumer in routine activities, and various food items are available that promote health benefits to a female population of consumers.

Moreover, many food products are in pre-packaged or ready-to-eat form and have generic ingredients. Preservatives and salts are used for economic reasons (to increase shelf life), with excess salt also used to enhance flavoring. Health-conscious people are limiting consumption of such foods or completely avoiding them due to the presence of excess fats/sugars, preservatives, or allergens which can impact their health in a negative manner.

While many restaurants and food outlets are providing healthy food options to their customers, in many situations the user requires a more specialized diet. In these cases, a high degree of personalization is required by the user/consumer as many factors must be considered. For example, the user/consumer may need a diet rich in calcium and low in sodium, while also needing to reduce overall caloric intake.

Therefore, a need has arisen for an improved method and/or system to address one or more of the above-mentioned disadvantages and improve the health of consumers by providing personalized meal portion(s) to consumers/users.

### BRIEF DESCRIPTION OF THE DRAWING(S)

FIG. 1 is a diagrammatic illustration of the system of the invention.

FIG. 2 schematically shows an exemplary flow diagram of a method of arranging delivery of a personalized meal portion using at least one processing device.

### DETAILED DESCRIPTION

Aspects of the present invention can be understood by reference to the figure and description set forth herein. However, the following descriptions, and numerous specific details thereof, are given by way of illustration and not of limitation. Many changes and modifications to the specific descriptions may be made without departing from the spirit and scope thereof, and the present invention includes all such modifications. It is to be understood that the disclosed embodiments may or may not overlap with each other. Thus, part of one embodiment, or specific embodiments thereof, may or may not fall within the ambit of another, or specific embodiments thereof, and vice versa.

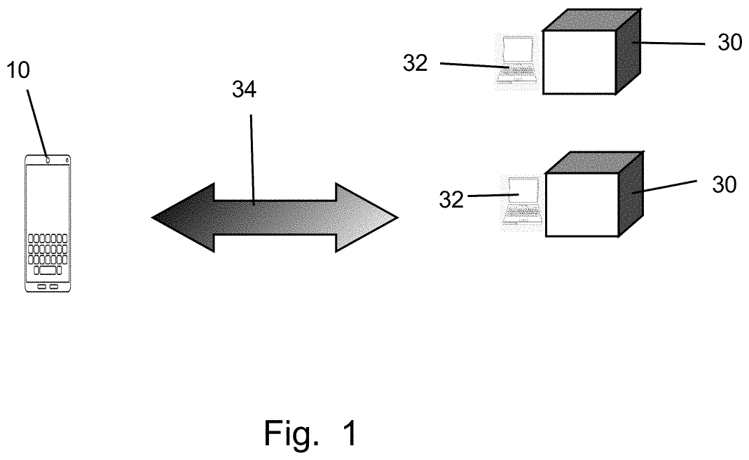

FIG. 1 diagrammatically shows the interconnection of the participants practicing the method of the invention. A user having an electronic processing device 10 with a display 12 can interface with an online portal 30 which delivers cooked or uncooked meal portions to users. A processing device or computer 32 associated with the portal 30, which computer 32 may be a server, or a series of “cloud” servers accessible via a network such as the internet 34, receives instructions from the device 10 over the network. The computer 32 is programmed with software for performing the function of the invention. The user's device 10 can download software from computer 32 or a dedicated third-party computer, the software generating a series of GUI driven menu interfaces allowing the user to communicate with computer 32 and effect the method of the invention as described below. A computer 36 or other device associated with a third party may also be used to affect the contents of the personalized meal. The third party could be a physician, a personal trainer, or other professional that gives nutritional advice to users.

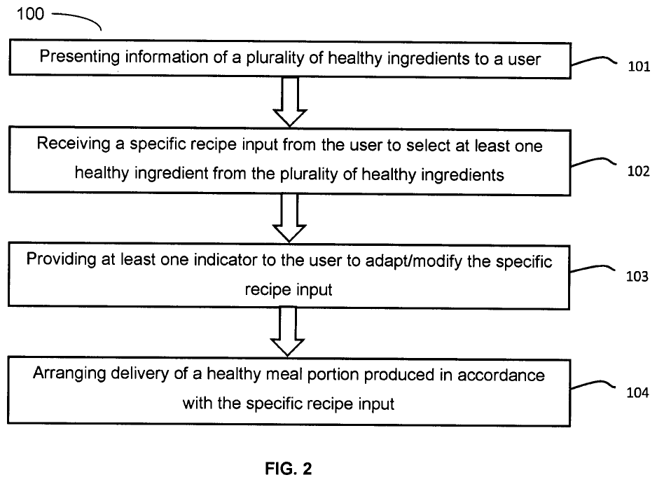

FIG. 2 shows an exemplary flow diagram of a method 100 of arranging delivery of a personalized or healthy meal portion using at least one processing device. The personalized meal portion can include both food and beverage items for consumption by users.

The method 100 includes a step 101 of presenting information pertinent to a plurality of ingredients to a user, the ingredients stored on or accessible by computing device 32. In other words, a list of ingredients specifically targeted to a specific user, is displayed on the user device 10. The ingredients, which may be called healthy ingredients, could include such things as foods high in fiber or antioxidants. The user can be a new user, a guest user, or a pre-registered user on an application, or another online portal related to ordering and delivery of meal portions.

The method 100 further includes a step 102 of receiving a specific recipe request or input from the user via device 10 to select at least one ingredient from the plurality of ingredients. The specific recipe input further comprises at least one of a quantity of the at least one ingredient selected by the user, at least one cooking instruction, and a size of the personalized meal portion. Therefore, the application stored on the user device 10 provides the user with a simple process of selecting one or more ingredients from the plurality of ingredients to personalize the healthy meal portion, as well as a method of cooking or preparing the meal portion.

The method 100 further includes a step 103 of providing at least one indicator to the user to adapt or modify the specific recipe input. The indicator is in the form of lists of alternatives to selected ingredients or a selected recipe, which alternatives may include substituting ingredients, cooking styles, or both. A user may opt to “select” one or more alternatives presented on display 12 using the well known select and click method on device 10.

The method 100 further includes a step 104 of arranging delivery of the healthy meal portion to the user, and actually delivering the meal portion. Thus, the output of the method 100 is a personalized healthy meal portion. It should be noted that the term meal portion can be an entire meal or just, e.g., a selected protein cooked or prepared using a desired method. The healthy meal portion is produced in accordance with the specific recipe input before delivery to the user.

According to another embodiment of the present invention, the healthy meal portion can be produced in accordance with the specific recipe input adapted according to the at least one indicator.

According to another embodiment of the present invention, the specific recipe input is adapted (finalized) upon receiving approval by the user. The changes to the recipe are finalized when approved by the user.

According to another embodiment of the present invention, the at least one indicator can be based on one or more of a profile of the user, an input of a professional, a recipe database, a user database, a nutritional content database, a calorie-meter, or a combination thereof.

The profile of the user can include specific information of the user corresponding to one or more static or dynamic fields such as, but not limited to, name, age, sex, demographic information, medical history, clinical requirements, preferences, eating and drinking habits or patterns, personalized diet programs and previous order history. In one specific example, the user can update static details of his/her profile on the application or the online portal 30 as a guest user or a registered user. In another specific example, the user can also update static details of his/her profile during creation of a personal/login account on the website/application or the online portal. Alternatively, specific information corresponding to at least some of the fields of the profile can also be updated or fetched on the basis of linking with a third-party online application on which the user has a registered account already. Examples of such online applications include, but are not limited to, an email application, a social networking application or a healthcare or fitness application). Furthermore, dynamic fields of the profile of the user get updated automatically according to the change of related events with time.

The input of the professional can include expert advice from a chef, a personal trainer, or a healthcare professional to make the meal more suitable or healthy for the specific user. For example, a user having a potassium deficiency and high blood pressure would need a meal high in potassium and low in sodium. The input of the professional can be further based on the specific information provided or updated in the profile of the user.

The recipe database, which is stored on or accessed by computing device 32, contains details of previous recipes saved by the user who is requesting delivery of the meal portion. The recipe database can contain details of previous recipes of other users as well. Therefore, each user will be able to save his/her recipes or also view recipes of other users (with permission) who also use the website/application/online portal related to ordering and delivery of meal portions. Additionally, the recipe database can include a standard recipe database containing standard recipes of various healthy or therapeutic foods/beverages or individual categories (for example, salads, soups, sandwiches, etc.). The recipe database can be updated dynamically with time.

The user database stores details of all the users including specific information from their respective profiles. Furthermore, the user database can include a mapping between different users based on any similarity or overlap in relevant fields of their respective profiles.

The nutritional content database stores values corresponding to nutritional content of each ingredient.

The calorie-meter counts the calorie of each ingredient and adds them up based on the specific recipe input provided and/or approved by the user.

According to one or more embodiment(s), the present invention provides a platform for those users interested in healthy eating to be able to find the right healthy ingredients and receive them as a portion of their own specific recipe by giving them options to customize the final healthy meal portion.

According to another embodiment of the present invention, the personalized meal portion can be produced by combining the at least one healthy ingredient with at least one basic ingredient in accordance with the specific recipe input. The at least one basic ingredient may be necessary to produce the healthy or personalized meal portion. For example, a particular type of bread is mandatory to prepare a particular type of sandwich. Therefore, that particular type of bread is the basic ingredient and therefore, selected by default irrespective of other healthy ingredient(s) selected by the user.

According to another embodiment of the present invention, the healthy meal portion can be produced by combining the at least one healthy ingredient with one or more other healthy or basic ingredients from the plurality of healthy or basic ingredients stored on or accessible to computing device 32 in accordance with the specific recipe input.

According to another embodiment of the present invention, delivery of the personalized meal portion is arranged according to one of a home-delivery arrangement and a pick-up arrangement. Both these delivery arrangements can be scheduled via the website/application/online portal.

According to another embodiment of the present invention, any user with a processing device 10 having a display 12 (for example, smartphone, tablet, laptop, computer, etc.) and internet 34 access who is looking for healthy food and beverages, can easily customize/personalize their salad/food/beverages and get a personalized meal delivered to them via the home-delivery arrangement or the pick-up arrangement.

According to another embodiment of the present invention, a system of arranging delivery of a healthy meal portion is also disclosed. The system further comprises the processing device 32 associated with the online portal; and at least one computer readable storage device storing instructions that, when executed by the processing device 32 cause the device to perform one or more steps disclosed in the method 100 of the present invention. In other words, the processing device 32 can be configured to perform one or more steps or methods disclosed in the method 100 of the present invention.

The term ‘processing device’ as used herein encompasses any device comprising a processor. The term ‘processor’ as used herein encompasses any electronic component which can execute program(s) or machine executable instructions. References to the processing/computing device having a processor should be interpreted as possibly containing more than one processor. The term ‘processing device’ should also be interpreted to possibly refer to a collection or network of processing/computing devices each comprising a processor. Many programs have their instructions performed by multiple processors that may be within the same processing device or which may be even distributed across multiple processing devices for providing a scalable or efficient processing of data. Illustrative examples of the processing device include, but not limited to, a personal computer, a desktop, a laptop, a mobile phone, a tablet, a computer, a server, a data center, etc.

One or more of the above-mentioned embodiments or method steps of the present invention can be automated completely or partially using one or more of components such as at least one processing device 10 and software. One or more steps to arrange delivery of the healthy meal portions can be implemented by use of these components.

It will be appreciated that the above description has described and illustrated specific embodiments and examples. However, the description is intended to cover any and all variations of various embodiments and examples of the invention. Combinations of the above embodiments/examples/arrangements not specifically described herein, will be apparent to those of skill in the art upon reviewing the above description. Therefore, it is intended that the disclosure is not limited to the particular embodiments/examples/arrangements disclosed, but that the invention will include all embodiments and arrangements falling within the scope of the appended claims.

1. A method of delivering of a personalized meal portion to a user from an online portal using at least one processing device, the method comprising:
presenting information of a plurality of healthy ingredients to the user;
receiving a specific recipe input from the user to select at least one healthy ingredient from the plurality of healthy ingredients, wherein the specific recipe input further comprises at least one of a quantity of the at least one healthy ingredient selected by the user, at least one cooking instruction, and a size of the personalized meal portion;
providing at least one indicator to the user to adapt the specific recipe input; and
delivering the personalized meal portion to the user, wherein the personalized meal portion delivered is produced in accordance with the specific recipe input.

2. The method of claim 1, wherein the personalized meal portion is produced in accordance with the specific recipe input adapted according to the at least one indicator.

3. The method of claim 2, wherein the specific recipe input is adapted upon receiving approval by the user.

4. The method of claim 1, wherein the at least one indicator is based on one or more of a profile of the user, an input of a professional, a recipe database, a user database, a nutritional content database, a calorie-meter, or a combination thereof.

5. The method of claim 1, wherein the personalized meal portion is produced by combining the at least one healthy ingredient with at least one basic ingredient in accordance with the specific recipe input.

6. The method of claim 1, wherein the personalized meal portion is produced by combining the at least one healthy ingredient with one or more other healthy ingredients from the plurality of healthy ingredients in accordance with the specific recipe input.

7. The method of claim 1, wherein delivery of the personalized meal portion is arranged according to one of a home-delivery arrangement and a pick-up arrangement.

8. A method of delivering of a personalized meal portion to a user having a networkable processing device from an online portal using at least one processing device, the method comprising:
presenting information of a plurality of ingredients to the user;
receiving a specific recipe request from the user to select at least one ingredient from the plurality of ingredients, wherein the specific recipe request further comprises at least one of a quantity of the at least one ingredient selected by the user, at least one cooking instruction, and a size of the personalized meal portion;
providing at least one option to the user to adapt the specific recipe request; and
delivering the personalized meal portion to the user, wherein the personalized meal portion delivered is produced in accordance with the specific recipe request.

9. The method of claim 8, wherein the personalized meal portion is produced in accordance with the specific recipe request adapted according to the at least one indicator.

10. The method of claim 8, wherein the specific recipe request is adapted upon receiving approval by the user.

11. The method of claim 8, wherein the at least one option is based on one or more of a profile of the user, an input of a professional, a recipe database, a user database, a nutritional content database, a calorie-meter, or a combination thereof.

12. The method of claim 8, wherein the personalized meal portion is produced by combining the at least one ingredient with at least one basic ingredient in accordance with the specific recipe request.

13. The method of claim 8, wherein the personalized meal portion is produced by combining the at least one ingredient with one or more other ingredients from the plurality of ingredients in accordance with the specific recipe request.

14. The method of claim 8, wherein delivery of the personalized meal portion is arranged according to one of a home-delivery arrangement and a pick-up arrangement.

15. A method of producing a personalized meal portion for a user having a networkable processing device from an online portal using at least one networkable processing device, the method comprising the steps of:
presenting information of a plurality of ingredients to the user;
receiving a specific recipe request from the user to select at least one ingredient from the plurality of ingredients, wherein the specific recipe request further comprises at least one of a quantity of the at least one ingredient selected by the user, at least one cooking instruction, and a size of the personalized meal portion;
providing at least one option to the user to adapt the specific recipe request; and
producing the personalized meal portion wherein the personalized meal portion is produced in accordance with the specific recipe request.

16. The method of claim 15, wherein the personalized meal portion is produced in accordance with the specific recipe request adapted according to the at least one option.

17. The method of claim 15, wherein the specific recipe request is adapted upon receiving approval by the user.

18. The method of claim 15, wherein the at least one option is based on one or more of a profile of the user, an input of a professional, a recipe database, a user database, a nutritional content database, a calorie-meter, or a combination thereof.

19. The method of claim 15, wherein the personalized meal portion is produced by combining the at least one ingredient with at least one basic ingredient in accordance with the specific recipe request.

20. The method of claim 15, wherein the personalized meal portion is produced by combining the at least one ingredient with one or more other ingredients from the plurality of ingredients in accordance with the specific recipe request.
